(19) United States
(12) Patent Application Publication
(10) Pub. No.: US 2017/0071779 A1
(43) Pub. Date: Mar. 16, 2017

(54) GARMENT WITH INTEGRATED REHABILITATION MECHANISM

(71) Applicant: Aimee EDGAR, West Hartford, CT (US)

(72) Inventor: Aimee EDGAR, West Hartford, CT (US)

(21) Appl. No.: 15/267,397

(22) Filed: Sep. 16, 2016

Related U.S. Application Data
(60) Provisional application No. 62/219,360, filed on Sep. 16, 2015.

(51) Int. Cl. A61F 5/37; A41D 27/20; A41D 27/10; A41D 15/00

(52) U.S. Cl. CPC A61F 5/3746; A41D 15/00; A41D 27/201; A41D 27/10; A41D 2300/322

(57) ABSTRACT

The present invention is directed to a garment that includes a section configured to cover at least a portion of a body of a user, and a rehabilitation mechanism integrated into the garment and operable between a normal wear configuration and a rehabilitation wear configuration. The rehabilitation mechanism includes a first fastener and a second fastener to allow for positioning of the rehabilitation mechanism between the normal wear configuration and the rehabilitation wear configuration.

### CROSS-REFERENCE TO RELATED APPLICATIONS

This application claims priority to U.S. Provisional Appl. No. 62/219,360 filed Sep. 16, 2015, which is hereby incorporated by reference in its entirety.

### BACKGROUND OF THE INVENTION

1. Field of the Invention The present invention is generally directed to garments that contain integrated rehabilitation mechanisms, and more particularly to shirts incorporating a convertible sling designed to allow for arm protection and healing.

2. Description of Related Art

Surgery and/or injury to parts of the body, such as joints, usually require some form of rehabilitation in order to retain almost full use of the affected body part. Generally, body parts are immobilized and/or restricted for a certain amount of time in order to allow the body part to heal, and reduce the likelihood of further damage to the body part. Typical rehabilitation devices designed for these purposes are conspicuous, and typical clothing worn by a patient for everyday use may not facilitate the measures necessary for proper rehabilitation. Therefore, what is needed are garments that are capable of performing a rehabilitation function, while at the same time able to be worn as typical garments as well.

### SUMMARY OF THE INVENTION

The present invention is designed to overcome the above noted limitations that are attendant upon the use of conventional garments and rehabilitation devices and, toward this end, it contemplates the provision of a novel garment incorporating an integrated rehabilitation mechanism.

It is an object of the present invention to provide clothing designed with a specific focus on rehabilitation that is adaptable, adjustable, comfortable, functional and fashionable.

It is another object of the present invention to provide shirts that appear to be regular shirts but have the ability to be morphed into a sling using snaps that are incorporated into the shirt design allowing for arm protection and healing, while at the same time being more comfortable for patients, thereby increasing patient compliance during recovery.

It is still another object of the present invention to provide dual-function shirts that can be worn without sling function for regular day-to-day wear, or can be transformed into a rehabilitation or sling function.

It is yet another object of the present invention to provide clothing designed to foster recovery on a daily basis utilizing various modalities, such as ice and compression.

It is another object of the present invention to provide garments that allow patients multiple options and choice regarding fabric type and style for their brace during recovery.

It is yet another object of the present invention to provide a garments that are configured to incorporate the recovery principles of rest, ice, compression, elevation, for example by providing shirts with the ability to reconfigure into a sling to effect rest and elevation, and shirts and pants with pockets for icing and compression.

According to an exemplary embodiment of the present invention a garment is provided that may include a section configured to cover at least a portion of a body of a user, and a rehabilitation mechanism integrated into the garment and operable between a normal wear configuration and a rehabilitation wear configuration.

In accordance with this or other exemplary embodiments of the present invention, the rehabilitation mechanism may include a first fastener and a second fastener to allow for positioning of the rehabilitation mechanism between the normal wear configuration and the rehabilitation wear configuration.

In accordance with this or other exemplary embodiments of the present invention, the rehabilitation mechanism may include at least one strap having the first fastener positioned thereon, and configured for connection to the second fastener, when the first fastener is connected to the second fastener the at least one strap is configured to position the rehabilitation mechanism in the rehabilitation wear configuration.

In accordance with this or other exemplary embodiments of the present invention, the rehabilitation mechanism may include a kangaroo pocket having the second fastener positioned thereon.

In accordance with this or other exemplary embodiments of the present invention, when the rehabilitation mechanism is in the rehabilitation wear configuration, the at least one strap is configured to provide support for the kangaroo pocket.

In accordance with this or other exemplary embodiments of the present invention, the at least one strap may include a first strap and a second strap, the first fastener is positioned on the first strap and a third fastener is positioned on the second strap, the kangaroo pocket has the second fastener and a fourth fastener positioned thereon, and the first fastener is configured for attachment to the third fastener and the second fastener is configured for attachment to the fourth fastener in order to position the rehabilitation mechanism into the rehabilitation wear configuration.

In accordance with this or other exemplary embodiments of the present invention, the at least one strap is integrated into the garment.

In accordance with this or other exemplary embodiments of the present invention, the at least one strap is removably attachable to the garment.

In accordance with this or other exemplary embodiments of the present invention, the garment may also include at least one storage pocket configured to hold the at least one strap when the rehabilitation mechanism is in the normal wear configuration.

In accordance with this or other exemplary embodiments of the present invention, the garment may include at least one sleeve, wherein one of the at least one sleeves has the second fastener positioned thereon.

In accordance with this or other exemplary embodiments of the present invention, the at least one sleeve comprises a first sleeve and a second sleeve, the first sleeve has the second fastener positioned therein, and the second sleeve has a third fastener positioned thereon, and wherein both of the second fastener and the third fastener are configured for attachment to the first fastener to allow use of either the first sleeve or the second sleeve to be able to positioned the rehabilitation mechanism in the rehabilitation wear configuration.

In accordance with this or other exemplary embodiments of the present invention, the rehabilitation mechanism may include a portion of the garment having at least a first fastener positioned thereon, and wherein the garment further comprises at least one second fastener positioned thereon.

In accordance with this or other exemplary embodiments of the present invention, the first fastener is configured for attachment to the second fastener for positioning the rehabilitation mechanism into the rehabilitation wear configuration.

### BRIEF DESCRIPTION OF THE SEVERAL VIEWS OF THE DRAWINGS

For a fuller understanding of the nature and object of the present invention, reference should be had to the following detailed description taken in connection with the accompanying drawings, in which:

FIG. 1A is a front view of an exemplary embodiment of a garment according to the present invention in a normal wear configuration;

FIG. 1B is a front view of an exemplary embodiment of a garment according to the present invention in a rehabilitation wear configuration;

FIG. 1C is a rear view of an exemplary embodiment of a garment according to the present invention in a normal wear configuration;

FIG. 1 is a front view of an alternative of the exemplary embodiment of the garment from FIGS. 1A-1C in a normal wear configuration;

FIG. 2 is a front view of an alternative of the exemplary embodiment of the garment from FIGS. 1A-1C in a rehabilitation wear configuration;

FIG. 3 is a front view of an alternative of the exemplary embodiment of the garment from FIGS. 1A-1C;

FIG. 4 is a front view of an alternative of the exemplary embodiment of the garment from FIGS. 1A-1C;

FIG. 5 is a front view of an alternative of the exemplary embodiment of the garment from FIGS. 1A-1C;

FIG. 6 is a front view of an alternative of the exemplary embodiment of the garment from FIGS. 1A-1C;

FIG. 7A is a front view of an exemplary embodiment of a garment according to the present invention in a rehabilitation wear configuration;

FIG. 7B is a front view of an exemplary embodiment of a garment according to the present invention in a normal wear configuration;

FIG. 7C is a rear view of an exemplary embodiment of a garment according to the present invention in a normal wear configuration;

FIG. 7 is a front view of an alternative of the exemplary embodiment of the garment from FIGS. 7A-7C;

FIG. 8 is a front view of an alternative of the exemplary embodiment of the garment from FIGS. 7A-7C;

FIG. 9 is a front view of an alternative of the exemplary embodiment of the garment from FIGS. 7A-7C;

FIG. 10 is a front view of an alternative of the exemplary embodiment of the garment from FIGS. 7A-7C;

FIG. 11A is a front view of an exemplary embodiment of a garment according to the present invention in a rehabilitation wear configuration;

FIG. 11B is a front view of an exemplary embodiment of a garment according to the present invention in a normal wear configuration;

FIG. 11C is a rear view of an exemplary embodiment of a garment according to the present invention in a normal wear configuration;

FIG. 11 is a front view of an alternative of the exemplary embodiment of the garment from FIGS. 11A-11C;

FIG. 12 is a front view of an alternative of the exemplary embodiment of the garment from FIGS. 11A-11C;

FIG. 13 is a back view of an alternative of the exemplary embodiment of the garment from FIGS. 11A-11C;

FIG. 14 is a front view of an alternative of the exemplary embodiment of the garment from FIGS. 11A-11C;

FIG. 15 is a front view of an alternative of the exemplary embodiment of the garment from

FIGS. 11A-11C;

FIG. 16 is a front view of another exemplary embodiment of a garment according to the present invention in a normal wear configuration;

FIG. 17 is a front view of the other exemplary embodiment of the garment according to the present invention in a rehabilitation wear configuration;

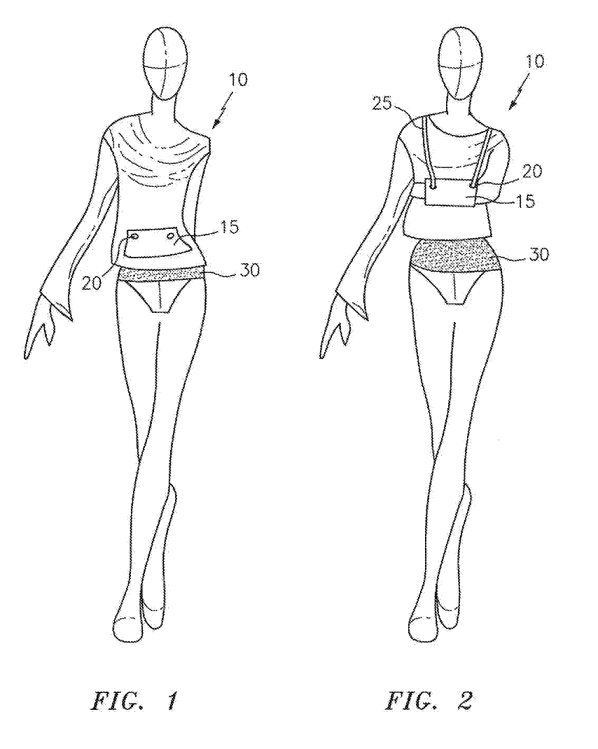

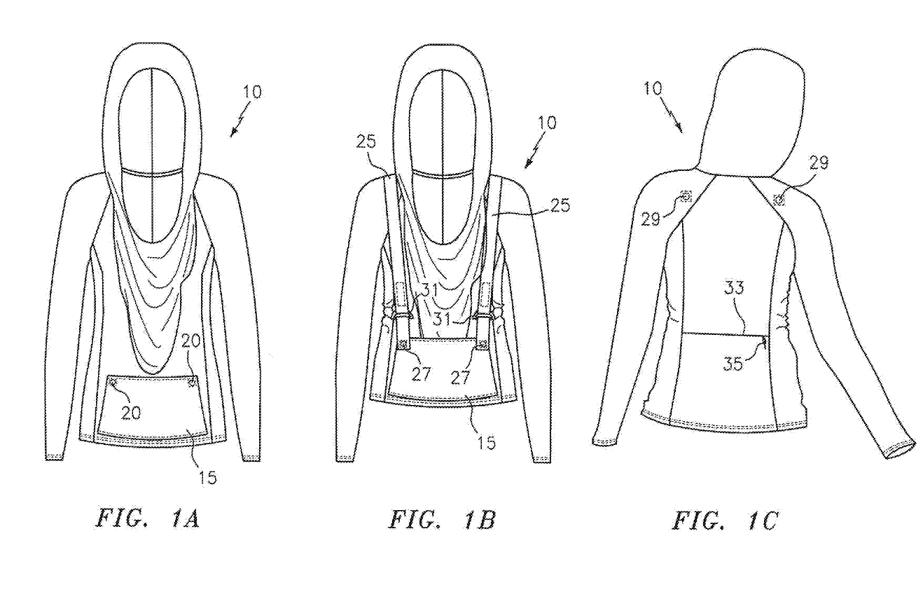

FIG. 18 is a front view of an alternative of the exemplary embodiment of the garment from FIGS. 1A-1C; and

FIG. 19 is a front view of an alternative of the exemplary embodiment of the garment from FIGS. 1A-1C.

### DETAILED DESCRIPTION OF THE INVENTION

The present invention now will be described more fully hereinafter with reference to the accompanying figures, in which exemplary embodiments of the invention are shown. The invention may, however, be embodied in many different forms and should not be construed as limited to the embodiments set forth herein.

Referring now to FIGS. 1A, 1B and 1C, therein illustrated is an exemplary embodiment of a garment, generally indicated by reference numeral 10, according to the present invention. The garment 10 includes an integrated rehabilitation mechanism, and is configurable between a normal wear configuration (FIGS. 1A and 1C) and a rehabilitation wear configuration (FIG. 1B). The garment 10 includes a kangaroo pocket 15 that is configured to act as a rehabilitation mechanism in the form of a sling, when the garment 10 is in the rehabilitation wear configuration. When the garment 10 in the normal wear configuration, the kangaroo pocket 15 may be configured to lay flat or substantially flat relative to the garment 10. The kangaroo pocket 15 includes at least one fastener 20, for example a snap, button, hook-and-pile fasteners or the like, that is configured for attachment to at least one strap 25 in order to lift the kangaroo pocket 15 and/or provide support for the kangaroo pocket 15. The at least one strap 25 has a fastener 27 corresponding to the fastener 20 on the kangaroo pocket, for example if a female snap is used as the fastener 20, then the at least one strap 25 would include a male snap as the corresponding fastener 27. The at least one strap 25 may also include a fastener (not shown) on another end of the strap 25 to secure the strap 25 to the garment 10, or the strap 25 may be integrally affixed to the garment 10, such as by sewing, adhesives or integrated fabric, without the need for additional fasteners. For example, as shown in FIG. 1C, the garment 10 may include one or more fasteners 29 positioned on the rear of the garment 10 to allow affixation of the other end of the one or more strap 25 to the rear of the garment 10. The one or more fasteners 29, as well as fasteners 20, 27 may be reinforced with additional support provided inside the garment 10. The at least one strap 25 may also be adjustable through the use of a cam buckle 31 in order to adjust the height at which the kangaroo pocket 15 may be used as a sling. The garment 10 may also include a storage pocket 33, which may be positioned on the back of the garment 10 to permit storage of the one or more strap 25, when the one or more strap 25 is not in use. The storage pocket 33 may be closed through the use of a closure mechanism 35, for example a zipper.

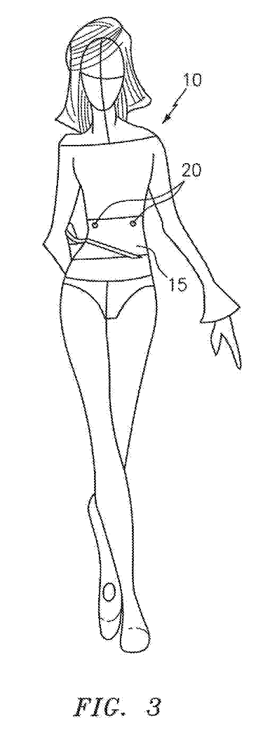

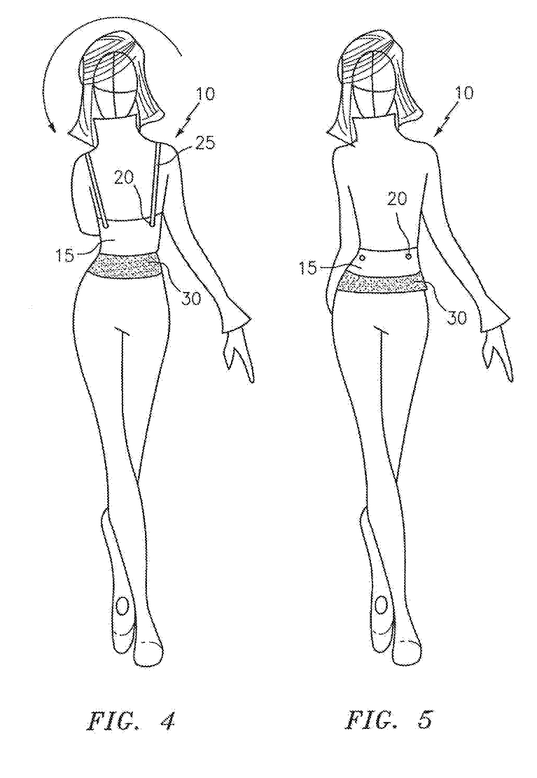

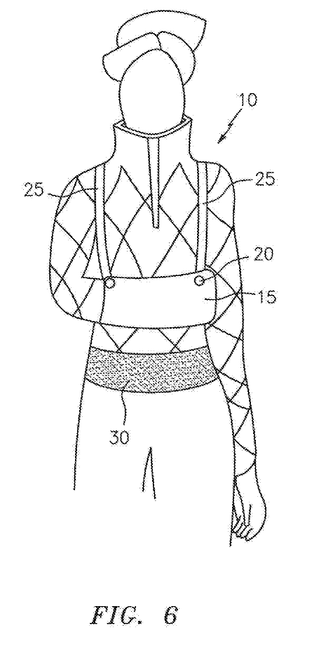

FIGS. 1-6 and 18-19 show alternatives for the garment 10 discussed above, and it is understood that the alternatives contain the same general components and function in the same manner as the garment discussed above with respect to FIGS. 1 and 2. In addition, to the components and functionality already discussed, the garment 10 may also include an integrated undershirt 30, so that as the garment 10 is lifted up so that the kangaroo pocket 15 can be used as a sling, the user's midsection is not exposed. Additionally, or alternatively the one or more straps 25 may be stored in a pocket (not shown) near the base of the garment 10 when not needed. It is understood that any of these features may be incorporated into any of the garments 10 shown in FIGS. 1A-1C, 3-6 and 18-19. In addition, to the components and functionality already discussed, the garment 10 shown in FIGS. 18-19 may also include a shoulder pocket 37 and/or shoulder opening that is configured to allow for inspection access to the shoulder area, insertion of ice or hot/cold compresses, insertion and/or retention of a compression bladder and insertion and/or retention of motion sensors. For example, the shoulder pocket 37 may be an external pouch on the garment 10 that allows for insertion and retention of items such as ice or heat packs. The shoulder pocket 37 may also be positioned internal to the garment, and then the shoulder pocket 37 is provided to access the pouch positioned on the interior of the garment. The should pocket 37 may be operable though the use of zippers, snaps, buttons, hook and loop fasteners or the like, and also provide access to the shoulder area for inspection of the progress of any healing or other post injury or surgery remnants. It is further understood that while FIGS. 18 and 19 shows the shoulder pocket/shoulder opening 37 positioned on one side of the garment 10, the present invention is not limited to positioning on any particular side of the garment and/or to the garment 10 only having a single shoulder pocket/shoulder opening 37. Furthermore, the garment 10 may include additional pockets/openings (not shown) positioned at other locations on the garment 10 to perform substantially similar functions as the shoulder pocket/shoulder opening 37. It is understood that any of these features may be incorporated into any of the garments 10 shown in FIGS. 1A-1C and 1-6.

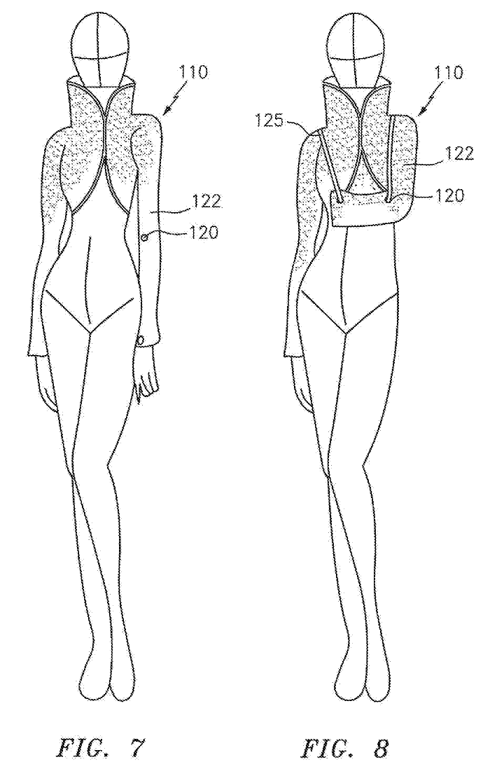

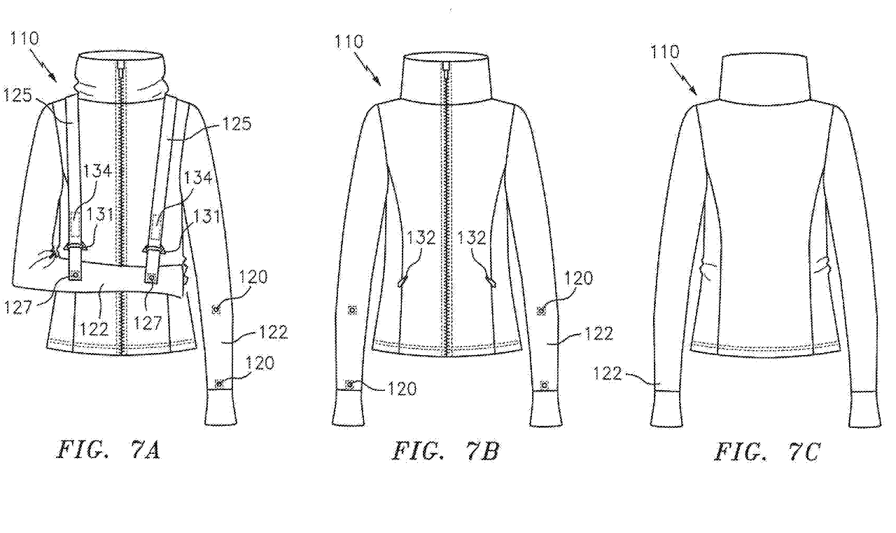

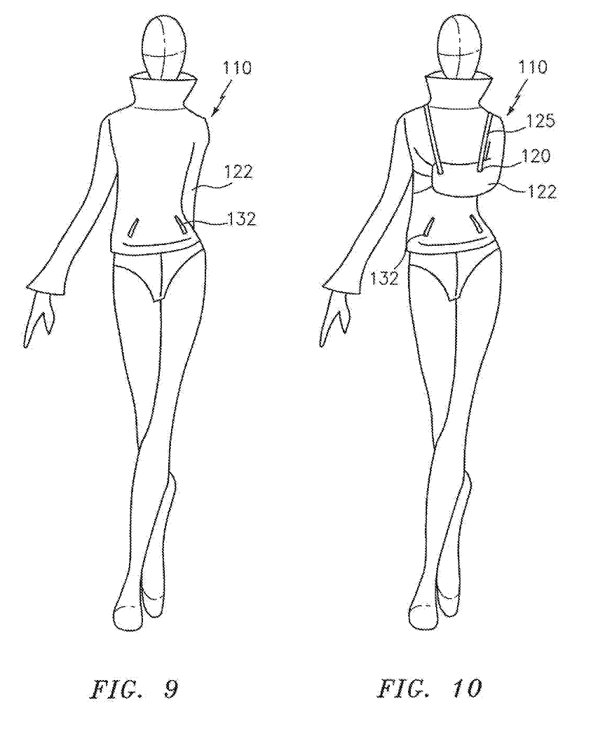

Referring now to FIGS. 7A, 7B and 7C, therein illustrated is an exemplary embodiment of a garment, generally indicated by reference numeral 110, according to the present invention. The garment 110 includes an integrated rehabilitation mechanism, and is configurable between a normal wear configuration (FIGS. 7B and 7C) and a rehabilitation wear configuration (FIG. 7A). The garment 110 includes at least one fastener 120, for example a snap, button, hook-and-pile fasteners or the like, positioned on a sleeve 122 of the garment 110, and the at least one fastener 120 is configured for attachment to at least one strap 125 in order to convert the sleeve 122 into a sling (FIG. 7A). Each sleeve 122 of the garment 110 may have the one or more fasteners 120 positioned thereon so that either sleeve 122 can be used for the rehabilitation wear configuration. The at least one strap 125 has a fastener 127 corresponding to the at least one fastener 120 on the sleeve 122, for example is a female snap is used as the fastener 120, then the fastener 127 of the at least one strap 125 would include a male snap. The strap 125 may be a single strap with one fastener 127 positioned at each end of the strap 125, and then in order to produce the rehabilitation wear configuration shown in FIG. 7A, the strap 125 is positioned about the neck and shoulders of the garment 110. Alternatively, one end of the strap 125 may be integrally affixed to the garment 110, such as by sewing, adhesives or integrated fabric, without the need for additional fasteners, in this case two straps 125 may be used, each of which may have an end affixed to the garment 110. The at least one strap 125 may also be adjustable through the use of one or more cam buckles 131 in order to adjust the height at which the sleeve 122 may be used as a sling. The at least one strap 125 may also include hook and loop fasteners 134 positioned thereon in order to facilitate adjustment of the strap 125 in addition to or in combination with the one or more cam buckles 131. The garment 110 may also include an integrated undershirt (not shown), so that the user's midsection is not exposed. The one or more straps 125 may be stored in a pocket 132 near the base of the garment 110 when not in use. The garment 110 may further include pockets (not shown) for storage of ice packets and/or for access to the injured shoulder for inspection. FIGS. 7-10 show alternatives for the garment 110 discussed above, and it is understood that the alternatives contain the same general components and function in the same manner as the garment discussed above with respect to FIGS. 7A-7C.

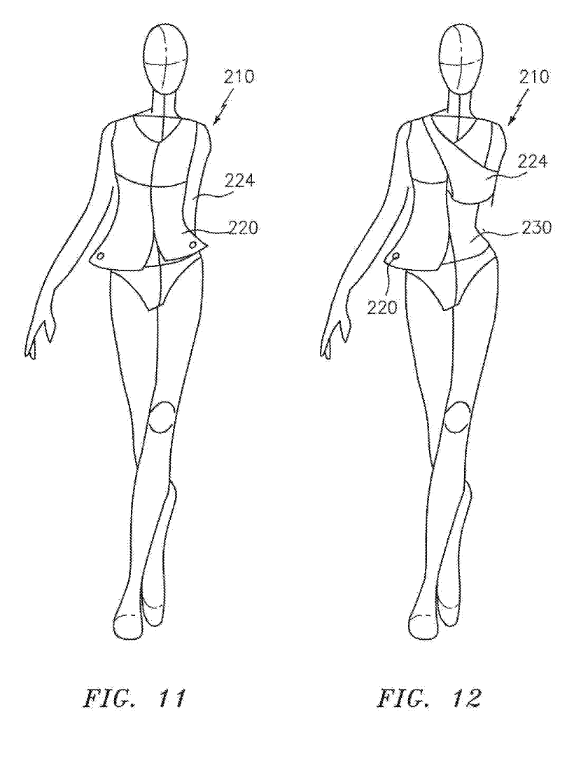

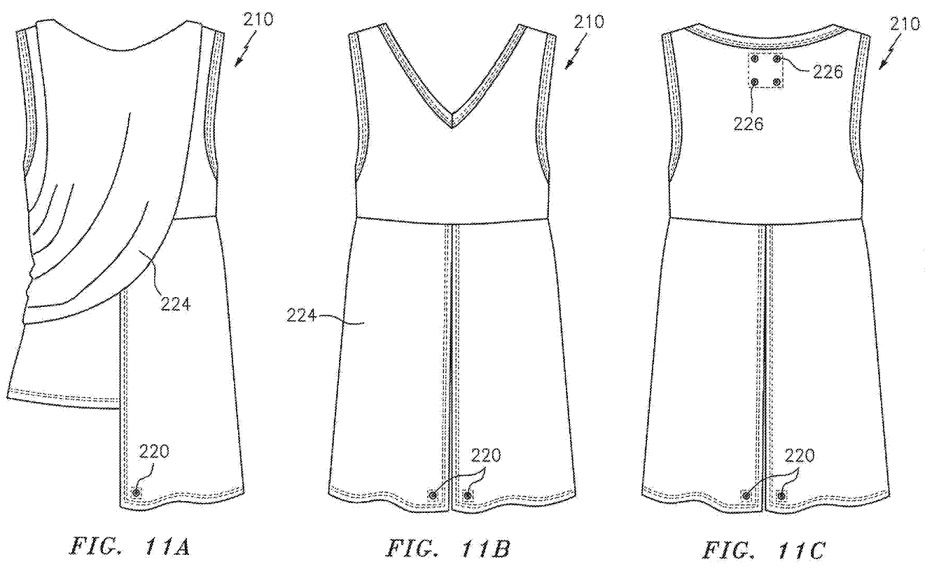

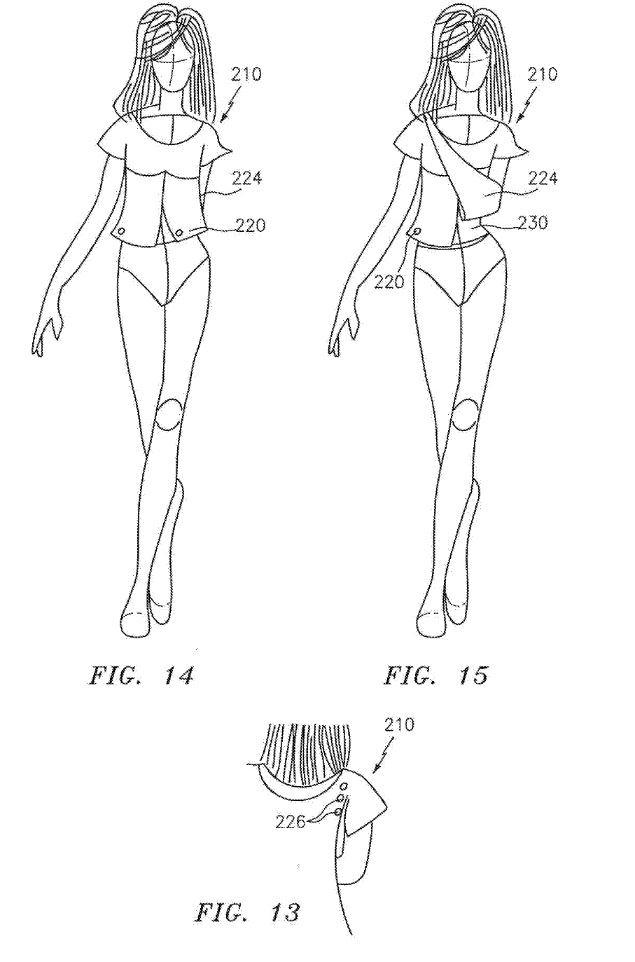

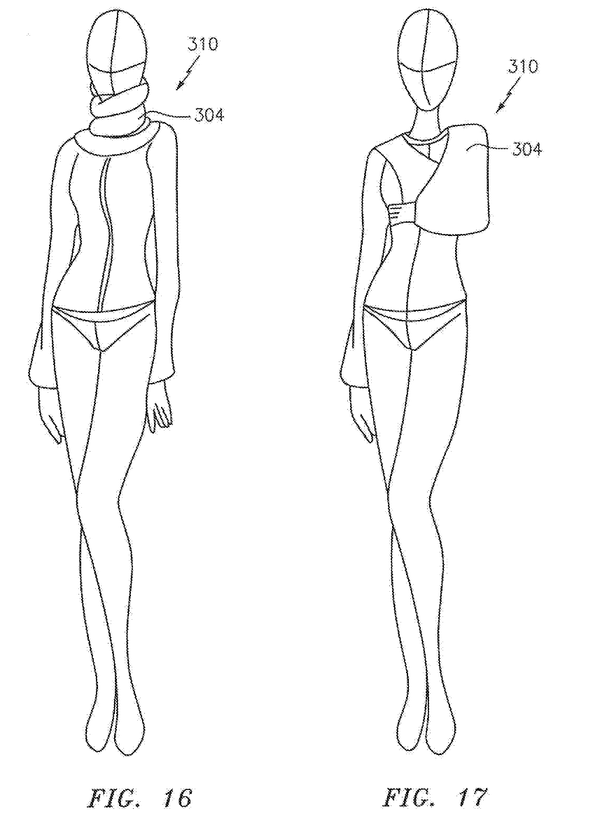

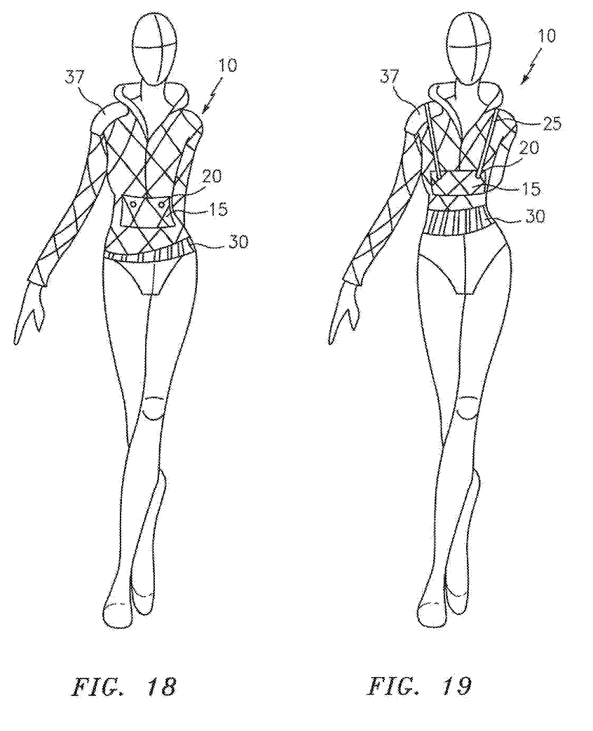

Referring now to FIGS. 11A, 11B and 11C, therein illustrated is an exemplary embodiment of a garment, generally indicated by reference numeral 210, according to the present invention. The garment 210 includes an integrated rehabilitation mechanism, and is configurable between a normal wear configuration (FIGS. 11B and 11C) and a rehabilitation wear configuration (FIG. 11A). The garment 210 includes at least one fastener 220, for example a snap, button, hook-and-pile fasteners or the like, positioned on a portion 224 of the garment 210, and the at least one fastener 220 is configured for attachment to a corresponding fastener 226 positioned on the back of the garment 220 in order to convert the portion 224 into a sling (FIG. 11A) to produce the rehabilitation wear configuration. The garment 210 may further include pockets (not shown) for storage of ice packets and/or for access to the injured shoulder for inspection. FIGS. 11-15 show alternatives for the garment 210 discussed above, and it is understood that the alternatives contain the same general components and function in the same manner as the garment discussed above with respect to FIGS. 11A-11C.

FIGS. 16-17 show another exemplary embodiment of the present invention, in which a garment 310 includes an integrated rehabilitation mechanism, and is configurable between a normal wear configuration (FIG. 16) and a rehabilitation wear configuration (FIG. 17). The garment 310 includes a scarf 304 that may be unraveled and attached to the garment 310 at least at two points to create a sling.

The garments according to the exemplary embodiments of the present invention may be made from any suitable fabrics depending upon the style, fit and/or function of the garments, and it is understood that the garments according to the present invention are not limited to any particular fabrics and/or materials.

It will thus be seen that the objects set forth above, among those made apparent from the preceding description, are efficiently attained and, since certain changes may be made in the above article without departing from the scope of this invention, it is intended that all matter contained in this disclosure or shown in the accompanying drawings, shall be interpreted, as illustrative and not in a limiting sense. It is to be understood that all of the present figures, and the accompanying narrative discussions of corresponding embodiments, do not purport to be completely rigorous treatments of the invention under consideration. It is to be understood that the above-described arrangements are only illustrative of the application of the principles of the present invention. Numerous modifications and alternative arrangements may be devised by those skilled in the art without departing from the scope of the present invention.

What is claimed is:

1. A garment, comprising:
a section configured to cover at least a portion of a body of a user; and
a rehabilitation mechanism integrated into the garment and operable between a normal wear configuration and a rehabilitation wear configuration;
wherein the rehabilitation mechanism comprises a first fastener and a second fastener to allow for positioning of the rehabilitation mechanism between the normal wear configuration and the rehabilitation wear configuration.

2. The garment according to claim 1, wherein the rehabilitation mechanism comprises at least one strap having the first fastener positioned thereon, and configured for connection to the second fastener, wherein when the first fastener is connected to the second fastener the at least one strap is configured to position the rehabilitation mechanism in the rehabilitation wear configuration.

3. The garment according to claim 2, wherein the rehabilitation mechanism comprises a kangaroo pocket having the second fastener positioned thereon.

4. The garment according to claim 3, wherein when the rehabilitation mechanism is in the rehabilitation wear configuration, the at least one strap is configured to provide support for the kangaroo pocket.

5. The garment according to claim 2, wherein the at least one strap comprises a first strap and a second strap, wherein the first fastener is positioned on the first strap and a third fastener is positioned on the second strap, wherein the kangaroo pocket has the second fastener and a fourth fastener positioned thereon, and wherein the first fastener is configured for attachment to the third fastener and the second fastener is configured for attachment to the fourth fastener in order to position the rehabilitation mechanism into the rehabilitation wear configuration.

6. The garment according to claim 2, wherein the at least one strap is integrated into the garment.

7. The garment according to claim 2, wherein the at least one strap is removably attachable to the garment.

8. The garment according to claim 2, further comprising at least one storage pocket configured to hold the at least one strap when the rehabilitation mechanism is in the normal wear configuration.

9. The garment according to claim 2, further comprising at least one sleeve, wherein one of the at least one sleeves has the second fastener positioned thereon.

10. The garment according to claim 9, wherein the at least one sleeve comprises a first sleeve and a second sleeve, the first sleeve has the second fastener positioned therein, and the second sleeve has a third fastener positioned thereon, and wherein both of the the second fastener and the third fastener are configured for attachment to the first fastener to allow use of either the first sleeve or the second sleeve to be able to positioned the rehabilitation mechanism in the rehabilitation wear configuration.

11. The garment according to claim 1, wherein the rehabilitation mechanism comprises a portion of the garment having at least a first fastener positioned thereon, and wherein the garment further comprises at least one second fastener positioned thereon.

12. The garment according to claim 11, wherein the first fastener is configured for attachment to the second fastener for positioning the rehabilitation mechanism into the rehabilitation wear configuration.
